(19) United States
(12) Patent Application Publication
(10) Pub. No.: US 2018/0362923 A1
(43) Pub. Date: Dec. 20, 2018

(54) METHOD FOR SEPARATING AND EXTRACTING HUC-MSC FROM WHARTON'S JELLY TISSUE OF UMBILICAL CORD

(71) Applicants: LEI GUO, Beijing (CN); CHENG LI, Beijing (CN)

(72) Inventor: LEI GUO, Beijing (CN)

(21) Appl. No.: 16/061,311

(22) PCT Filed: Dec. 11, 2015
(86) PCT No.: PCT/CN2015/097147

§ 371 Date: Jun. 11, 2018

(51) Int. Cl. C12N 5/073; C12N 9/64; C12N 5/0775; C12N 5/00

(52) U.S. Cl. CPC C12N 5/0605; C12N 9/6491; C12N 5/0668; C12N 5/0037; C12N 2501/115; C12N 2501/70; C12Y 304/24007; C12N 2501/603; C12N 2501/605; C12N 2501/602; C12N 2500/44

(57) ABSTRACT

Provided is a method for rapidly separating and extracting human umbilical cord mesenchymal stem cells (hUC-MSC). The method comprises the following steps: taking freshly collected healthy neonatal umbilical cord tissue, and after removing the blood vessels, bluntly dissecting the Wharton's jelly, mechanically pulverising same, and treating with erythrocyte lysate for 3 minutes; carrying out type IV collagenase digestion, and after sieving through a 100-200 mesh sieve, carrying out suspension culture in a serum-free culture medium, replacing the liquid every 3-5 days; taking the supernatant to detect cell contamination, and waiting for the adherence rate to reach 30-70%; carrying out trypsin digestion, collecting the cells by centrifugation, and carrying out passage amplification, until the rate of confluence of the cells reaches over 90%; collecting the cells for cryopreservation, and detecting the biological characteristics of the hUC-MSC.

### TECHNICAL FIELD

The invention relates to an efficient and rapid method for separating and extracting mesenchymal stem cells from umbilical cord, especially a method for separating mesenchymal stem cells from Wharton's jelly tissue of fresh umbilical cord.

### BACKGROUND OF THE INVENTION

Mesenchymal stem cell (MSC), with high self-renewal capacity and multiple differentiation potential, derives from the mesoderm and ectoderm of early development stage and exists extensively in the connective tissues and mesenchymes in organs of the whole body. By now, previous studies have indicated that different culture methods can make MSCs express neuronal phenotype, islet like cell phenotype or endothelial cell phenotype, or express cardiac myocyte markers, thus revealing the broad clinical application prospect of MSC in immunosuppression, regulation of endocrine system, modulation of nervous system and improvement in cardiovascular function. Therefore, MSCs have gradually become a valuable source of cells in the field of cell therapy and gene therapy. In particular, compared with MSCs from other sources, human Umbilical Cord mesenchymal stem cells (hUC-MSCs), which derive from human umbilical cord, have many advantages, such as, they have a simple growth environment and can be extracted conveniently, have strong capability of proliferation and differentiation, and low immunogenicity, and have no ethical problems.

In view of the multi-directional differentiation potential of hUC-MSC and its potential application in the field of disease treatment, there will be a broad prospect in the storage business of hUC-MSC in the domestic market. However, how to guarantee an efficient and standard rate of separating and extracting stem cells and ensure the quality of stem cells so as to meet the future clinical need for stem cell transplantation is an urgent problem to be solved in stem cell storage in China.

Now, most of hUC-MSCs are prepared by enzyme digestion and tissue adherence method. Numerous methods for separating and expanding hUC-MSCs have been reported at home and abroad; however, most of them involve rather complicated steps. Therefore, there are still certain difficulties in quickly extracting high purity hUC-MSCs in a large amount.

### SUMMARY OF THE INVENTION

Therefore, the purpose of the invention is to provide an efficient and rapid method for separating and extracting hUC-MSCs to meet the needs in this field.

Another purpose of the invention is to provide hUC-MSCs prepared by the method of the present invention.

Specifically, in view of the present situation of extraction of human umbilical cord mesenchymal stem cells at home and abroad, the present invention employs red blood cell lysis buffer to treat red blood cells which have a great influence on the primary growth of hUC-MSCs and employs collagenase digestion to shorten the duration of the primary culture, so as to achieve high and efficient extraction of high purity hUC-MSCs. Furthermore, the method of present invention utilizes a serum-free culture system which is more beneficial to the clinical transformation and application of the stem cells.

Technical solutions of the invention are as follows.

In one aspect, the present invention provides a method for separating and extracting mesenchymal stem cells from freshly isolated Wharton's jelly tissue of umbilical cord, which is a method for separating and culturing umbilical cord mesenchymal stem cells, the method comprising: using red blood cell lysis buffer and collagenase to treat the Wharton's jelly tissue.

The red blood cell lysis buffer used in the present invention is an aqueous solution comprising NH₄Cl and Na₂-EDTA, preferably an aqueous solution comprising 1-20 g/L NH₄Cl and 0.05-0.2 mM Na₂-EDTA, more preferably an aqueous solution comprising 5-10 g/L NH₄Cl and 0.1 mM Na₂-EDTA, pH 7.2-7.4. Before use, the red blood cell lysis buffer is filtered through 0.22 μm microfiltration membrane and equilibrated to room temperature.

The collagenase used in the present invention is type IV collagenase, preferably a digestion solution comprising type IV collagenase, more preferably D-Hank's comprising type IV collagenase, hyaluronidase, DNase and serum substitute, and further more preferably D-Hank's comprising 1% type IV collagenase, 0.5% hyaluronidase, 300 U/ml DNase and 2% serum substitute. The percentages are percentages by weight.

The method of the present invention specifically comprises: cutting the obtained Wharton's jelly tissue into tissue blocks, adding the red blood cell lysis buffer with a volume of 1-3 times the volume of the tissue blocks, and treating the tissue blocks with the buffer at room temperature for 2-5 minutes; and, adding the digestion solution comprising type IV collagenase into the treated tissue blocks for further treatment.

Preferably, the method of the present invention further comprises: after digestion by collagenase, culturing with serum-free medium for mesenchymal stem cells to obtain primary mesenchymal stem cells.

The serum-free medium for mesenchymal stem cells used in the present invention comprises a-MEM/DMEM-F12, β-mercapto ethanol, non-essential amino acids, recombinant human basic fibroblast growth factor (b-FGF) and serum substitute.

Preferably, the serum-free medium for mesenchymal stem cells comprises 0.05-0.2 parts by volume of β-mercapto ethanol, 0.5-2 parts by volume of an aqueous solution of non-essential amino acids, 8-12 parts by volume of serum substitute, 85-95 parts by volume of a-MEM/DMEM-F12 and recombinant human basic fibroblast growth factor at a final concentration of 5-15 ng/ml, wherein the aqueous solution of non-essential amino acids comprises glycine, alanine, L-asparagine, L-aspartic acid, glutamic acid, proline and serine each at a concentration of 8-12 mM; more preferably, the serum-free medium for mesenchymal stem cells comprises 0.1 parts by volume of β-mercapto ethanol, 1 part by volume of the aqueous solution of non-essential amino acids, 10 parts by volume of the serum substitute, 89 parts by volume of a-MEM/DMEM-F12 and the recombinant human basic fibroblast growth factor at a final concentration of 10 ng/ml; most preferably, the serum-free medium for mesenchymal stem cells consists of a-MEM/DMEM-F12, β-mercapto ethanol, the aqueous solution of non-essential amino acids, the recombinant human basic fibroblast growth factor and the serum substitute.

According to particular embodiments of the present application, the product available from Gibco under Catalog No. 11140 can be used as the aqueous solution of non-essential amino acids.

According to particular embodiments of the present application, the KnockOut™ Serum Replacement available from Gibco under Catalog No. 10828-010 can be used as the serum substitute.

Preferably, the method of the present invention specifically comprises: cutting the obtained Wharton's jelly tissue of umbilical cord into tissue blocks each in size of 1-3 mm³, adding the red blood cell lysis buffer with a volume of 1.5-2 times the volume of the tissue blocks, and treating the tissue blocks with the buffer at room temperature for 2-5 minutes; and into the treated tissue blocks, adding the digestion solution comprising type IV collagenase with a volume of 2-3 times the volume of the treated tissue blocks, and performing digestion at 37° C., 5% CO₂ for 8-12 hours.

Preferably, the method of the present invention further comprises: inoculating obtained cells in the serum-free medium for mesenchymal stem cells at a density of 1-5×10⁴ cells/cm² culture dish area and culturing at 37° C., 5% CO₂, during which the medium is replaced with fresh medium every 3-4 days.

Preferably, the method comprises the following steps:

(1) Pretreatment of Umbilical Cord Tissue:

Cutting fresh umbilical cord into segments, removing intravascular blood, longitudinally cutting each segment, eliminating umbilical artery and umbilical vein, bluntly dissecting the Wharton's jelly tissue and washing the same with PBS;

(2) Treatment with the Red Blood Cell Lysis Buffer:

Cutting the Wharton's jelly tissue obtained by step (1) into tissue blocks, adding the red blood cell lysis buffer to treat the tissue blocks, collecting the treated tissue blocks by centrifugation and washing them with PBS;

(3) Digestion with Collagenase:

Adding the digestion solution comprising type IV collagenase into the treated tissue blocks obtained by step (2) for further treatment, diluting by adding PBS, and collecting cells by filtration through sterile sieve;

(4) Primary Culture:

Culturing the cells obtained by step (3) in the serum-free medium for mesenchymal stem cells to obtain primary mesenchymal stem cells.

Preferably, the method further comprises the following steps:

(5) Supernatant Detection:

Taking the supernatant of cell culture obtained by step (4), and detecting one or more selected from the group consisting of hepatitis A, hepatitis B, hepatitis C, syphilis, human immunodeficiency virus, mycoplasma, chlamydia and endotoxin;

(6) Passage Culture:

Selecting the cultured cells with negative results in step (5), collecting cells by centrifugation after trypsin digestion, passage culturing, and collecting the cells or continuing passage;

(7) Cell Detection:

Taking the cells cultured in step (6), and detecting one or more selected from the group consisting of differentiation, cell activity, cell purity, cell contamination and proliferation profile.

Preferably, the step (1) includes: cutting the fresh umbilical cord into segments each of 2-3 cm in length, removing the intravascular blood, longitudinally cutting each segment, eliminating the umbilical artery and the umbilical vein, bluntly dissecting the Wharton's jelly tissue, adding PBS with a volume of 1.5-2 times the volume of the tissue, and washing the tissue by shaking slightly.

Preferably, the step (2) includes:

Cutting the washed Wharton's jelly tissue of umbilical cord into tissue blocks each in size of 1-3 mm³, adding the red blood cell lysis buffer with a volume of 1.5-2 times the volume of the tissue blocks, treating the tissue blocks with the buffer at room temperature for 2-5 minutes, collecting the treated tissue blocks by centrifugation, and washing 2-3 times with PBS; wherein the centrifugation preferably is performed at 1000-1200 rpm, 4° C. for 6 minutes.

Preferably, the step (3) includes:

Adding the digestion solution comprising type IV collagenase with the volume of 2-3 times the volume of the treated tissue blocks, performing digestion at 37° C., 5% CO₂ for 8-12 hours, and diluting by adding PBS; and, filtering through a 100 or 200 mesh sterile sieve, collecting filtrate, washing with PBS, and performing centrifugation to obtain cells; wherein the centrifugation preferably is performed at 1000-1200 rpm, 4° C. for 6 minutes.

Preferably, the step (4) includes:

Inoculating the cells obtained by step (3) in the serum-free medium for mesenchymal stem cells at a density of 1-5×10⁴ cells/cm² culture dish area and culturing at 37° C., 5% CO₂, during which the medium is replaced with fresh medium every 3-4 days.

Preferably, the step (5) includes:

Taking the supernatant of cell culture obtained by step (4) and detecting all of the followings: hepatitis A, hepatitis B, hepatitis C, syphilis, human immunodeficiency virus, mycoplasma, chlamydia and endotoxin.

Preferably, the step (6) includes:

Selecting the cultured cells with all negative results in step (5), performing trypsin digestion at 30-60% confluence, collecting cells by centrifugation, passage culturing to reach above 90% confluence, and collecting the cells for freezing conservation or continuing passage culture; wherein preferably, the trypsin is used at a mass percent concentration of 0.125%, and the digestion is performed for 1-2 minutes while patting the side walls of culture dish or culture flask used; and wherein preferably, the centrifugation is performed at 1000-1200 rpm, 4° C. for 6 minutes.

Preferably, the collected cells are freezing conserved in liquid nitrogen at −196° C. at a density of 2-3×10⁶ cells/ml; or preferably, the collected cells are passage cultured in a rate of 1:3-1:4.

Preferably, the step (7) includes:

Taking the cells cultured in step (6), and detecting all of the followings: differentiation, cell activity, cell purity, cell contamination and proliferation profile.

Preferably, prior to the step (1), the method further comprises washing, preserving and pre-processing the umbilical cord, preferably comprising:

collecting aseptically umbilical cord tissue from a healthy newborn by natural or cesarean section delivery, putting the umbilical cord tissue into preservation and transportion solution for umbilical cord after surface washing with sterile saline, preferably transporting the umbilical cordin ice to a clean cell laboratory within 6 hours; and before use, washing the fresh umbilical cord 2-3 times with 75% aqueous ethanol, then 3-5 times with sterile saline.

Preferably, the preservation and transportion solution for umbilical cord is magnesium and calcium free D-Hank's comprising penicillin sodium, streptomycin sulfate, gentamicin and amphotericin B for injection; more preferably, the concentration of each of penicillin sodium, streptomycin sulfate and gentamicin is 100-200 U/ml, preferably 150 U/ml, and the concentration of amphotericin B is 200-400 U/ml, preferably 300 U/ml.

According to particular embodiments of the present invention, the method for separating and extracting mesenchymal stem cells from Wharton's jelly tissue of freshly isolated umbilical cord comprises following steps:

(1) collecting and transporting the sample: collecting aseptically umbilical cord tissue sample from a healthy neoborn by natural or cesarean section delivery, putting the sample into the preservation and transportion solution for umbilical cord, and transporting in ice;

(2) Washing and sterilizing the umbilical cord sample: putting the fresh umbilical cord sample into a 50 ml aseptic centrifuge tube, washing the sample 2-3 times with 75% aqueous ethanol, then 3-5 times with sterile saline;

(3) Pre-processing the umbilical cord sample: cutting the umbilical cord into segments each of 2-3 cm in length with ophthalmic scissors, removing the intravascular blood with tweezers, longitudinally cutting each segment, eliminating two umbilical arteries and one umbilical vein with hemostatic forceps, bluntly dissecting the Wharton's jelly tissue, putting the Wharton's jelly tissue into a 50 ml centrifuge tube, adding PBS with a volume of 1.5-2 times the volume of the tissue, and washing the tissue by shaking slightly;

(4) Treating with the red blood cell lysis buffer: transferring PBS washed Wharton's jelly tissue to a clean and sterile 100 mm culture dish, cutting the tissue into tissue blocks each in size of 1-3 mm³, transferring minced cord tissue into a centrifuge tube, adding the red blood cell lysis buffer with a volume of 1.5-2 times the volume of the tissue blocks; treating the tissue blocks with the buffer at room temperature for 2-5 minutes, collecting the tissue blocks by centrifugation; and washing 2-3 times with PBS;

(5) Digesting with type IV collagenase: adding the digestion solution comprising type IV collagenase with a volume of 2-3 times the volume of the tissue blocks, transferring into an incubator at 37° C., 5% CO₂, 8-12 hours later transferring out of the incubator to a clean bench, and diluting by adding PBS; and filtering through a 100 or 200 mesh sterile sieve, collecting filtrate to a 50 ml centrifuge tube, washing with PBS, and performing centrifugation to remove residual enzyme;

(6) Primary culturing hUC-MSCs: flicking cell pellets on the bottom of the centrifuge tube, adding serum-free medium for mesenchymal stem cells to give a cell suspension, transferring the cell suspension to a 100 mm culture dish, and putting the culture dish into an incubator at 37° C. 5% CO₂ for culture, during which the medium is replaced with fresh medium every 3 days;

(7) Taking the supernatant of cell culture obtained by step (6) and detecting all of the followings: hepatitis A, hepatitis B, hepatitis C, syphilis, human immunodeficiency virus, mycoplasma, chlamydia and endotoxin;

(8) Passage culturing the hUC-MSC: performing trypsin digestion at 60-80% confluence in the dish, collecting cells by centrifugation and discarding the supernatant, re-inoculating the cells to a T75 cell culture flask for further passage culture to reach above 90% confluence, and collecting the cells for freezing conservation or continuing passage culture;

(9) Detecting all of the followings of the hUC-MSCs obtained by step (8): differentiation, cell activity, cell purity, cell contamination and proliferation profile.

In the methods mentioned above, the sample is fresh umbilical cord tissue.

In the methods mentioned above, the preservation and transportion solution for umbilical cord is a magnesium and calcium free D-Hank's comprising penicillin sodium, streptomycin sulfate, gentamicin and amphotericin B for injection prepared immediately before use, wherein the concentration of each of penicillin sodium, streptomycin sulfate and gentamicin is 100-200 U/ml, and the concentration of amphotericin B is 300 U/ml; and 40-60 mL of the preservation and transportion solution is packed in a sterile sample bottle and sealed by sealing film.

In the methods mentioned above, the medium for the mesenchymal stem cells is serum-free medium, which comprises 0.1 parts by volume of β-mercapto ethanol, 1 part by volume of the aqueous solution of non-essential amino acids, 10 parts by volume of serum substitute, 89 parts by volume of a-MEM/DMEM-F12 and b-FGF at a final concentration of 10 ng/ml.

The red blood cell lysis buffer is an aqueous solution comprising 5-10 g/L NH₄Cl and 0.1 mmol/L Na₂-EDTA, pH 7.2-7.4, which is filtered through 0.22 Lm microfiltration membrane and equilibrated to room temperature before use.

The digestion solution is D-Hank's comprising 1% type IV collagenase, 0.5% hyaluronidase, 300 U/ml DNase and 2% serum substitute.

Furthermore, in step (6) the cells are inoculated at a density of 2-3×10⁴ cells/cm².

In the step (8), the trypsin has a concentration of 0.125%, and the digestion is performed for 1-2 minutes while patting the side walls of culture dishes or culture flasks used; the passage ratio is 1:3-1:4; and the freezing conservation is carried out with 2-3×10⁶ cells reserved in 1 ml freezing conservation solution.

In another aspect, the present invention further provides the hUC-MSCs prepared by the methods mentioned above.

Preferably, the hUC-MSCs have characteristics as follows:

(1) Adhering to plastic container(s), appearing as spindle-shape and growing in whorls;

(2) Ratio of CD29, CD44, CD73, CD90, CD105 or HLA-ABC positive cells greater than 99%; and ratio of CD45, CD34 or HLA-DR positive cells less than 1.0%;

(3) Capable of being induced to differentiate into osteogenic cells and adipogenic cells in vitro;

(4) Ratio of viable cells detected above 99%;

(5) Having a typical “S type” growth curve; and

(6) Expressing pluripotency genes which are one or more selected from the group consisting of SSEA-4, OCT-4, NANOG and SOX-2.

In a further aspect, the present invention provides the use of the red blood cell lysis buffer, the digestion solution comprising type IV collagenase and/or the serum-free medium for mesenchymal stem cells in the preparation of an agent for separating and culturing mesenchymal stem cells.

In a yet another aspect, the present invention provides a kit for separating and culturing mesenchymal stem cells, which comprises the red blood cell lysis buffer, the digestion solution comprising type IV collagenase and/or the serum-free medium for the mesenchymal stem cells of the present invention.

The present invention provides a method for extracting mesenchymal stem cells from umbilical cord employing collagenase digestion assisted by the red blood cell lysis buffer, wherein the red blood cell lysis buffer is capable of eliminating red blood cells which have a great influence on growth of the primary hUC-MSCs, thereby largely improving the purity of the extracted hUC-MSCs; meanwhile, collagenase digestion significantly shortens the culture duration for the primary stem cells; thus, the method of the present invention has achieved good results. Overall, the exacting method of the present invention can harvest primary cells within 6-8 days, and the cell purity reaches above 99% after just two passages. Furthermore, the method of the present invention is easy to carry out with a low cost, and the serum-free medium employed has a well-defined composition, thereby avoiding instability of the cell growth during the culture due to batch difference of the serum, and excluding the possibility of spreading xenogeneic pathogens. The serum-free culture based on the said medium ensures the hUC-MSCs have higher safety and brighter prospect of application.

Results of flow cytometry detection, viability determination, differentiation identification and pluripotency gene analysis have shown that the mesenchymal stem cells obtained by the method of present invention have high viability and purity, and strong differentiation capability, and the cell bank established with the stem cells can be directly utilized in scientific research and clinical adjuvant treatment.

### BRIEF DESCRIPTION OF THE FIGURES

Now the implementations of the present invention will be described in detail in conjunction with accompanying drawings, in which:

FIG. 1 shows cell images during the screening of culture medium components, in which panel 1A shows cell morphology 48 hours after cells were inoculated in a medium comprising a low concentration of serum substitute, panel 1B shows cell morphology 24 hours after cells were inoculated in a medium comprising a high concentration of serum substitute, panel 1C shows cell morphology 24 hours after cells were inoculated in medium comprising a low concentration of bFGF, and panel 1D shows cell morphology after cells were passage cultured in a medium comprising a high concentration of bFGF.

FIG. 2 shows the cell morphology of the cultured hUC-MSC, in which panel 2A shows initial confluence state of the primary cells, panel 2B shows the fibrous cell morphology of cultured cells after passage, and panel 2C shows the cell morphology of cells without using red blood cell lysis buffer.

FIG. 3 (panels 3A to 3I) shows the analysis results of cell surface molecules of the obtained hUC-MSCs by flow cytometry, indicating that the hUC-MSCs expressed CD29, CD44, CD73, CD90, CD105 or HLA-ABC with ratio of positive cells greater than 99.0%, while did not express CD45, CD34 or HLA-DR with ratio of positive cells less than 1.0%.

FIG. 4 shows the analysis results of the cell viability and growth profile of the obtained hUC-MSCs by Vi-Cell cell vitality analyzer, in which panel 4A shows the real-time viability analysis of the hUC-MSCs, panel 4B shows the growth curve of the hUC-MSCs, panel 4C shows the diameter distribution of the hUC-MSCs, and panel 4D shows the roundness distribution of the hUC-MSCs after digestion. The results indicated that the viability of the hUC-MSCs was above 99.7% with a diameter distribution of 9-15 μm, and had proliferation profile characterized by latent stage, logarithmic growth stage and platform stage.

FIG. 5 shows the directed induced differentiation of the obtained hUC-MSCs into osteogenic cells or adipogenic cells, in which panel 5A shows dark red compounds produced by chromogenic reaction between alizarin red and calcium nodules during osteogenesis, and panel 5B shows the specific oil red O staining of the fat droplets in adipogenic cells.

FIG. 6 shows the RT-PCR analysis result of the pluripotency genes in the obtained hUC-MSCs at transcriptional level, with Lane M: DNA molecular weight marker; Lane 1: internal reference gene β-Actin; Lane 2: NANOG; Lane 3: OCT-4; Lane 4: SOX-2; and Lane 5: SSEA-4.

FIG. 7 shows analysis results of hUC-MSC pluripotency specific proteins by immunofluorescence staining.

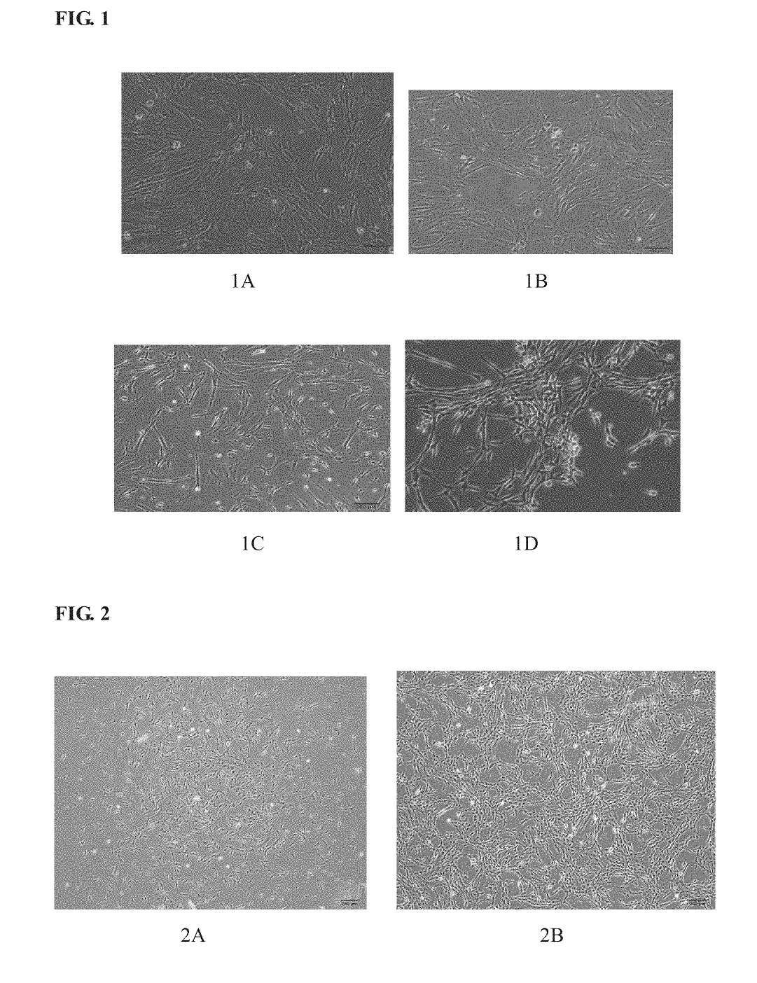

### DETAILED DESCRIPTION OF PREFERRED EMBODIMENTS

The present invention will be further described in detail in conjunction with following embodiments, and the Examples are provided here only to elaborate the invention but not to be construed as limiting the scope of the invention.

The procedures without specified conditions shall be carried out in accordance with conventional conditions in the field the invention belongs to or recommended conditions by the supplier of the instruments and reagents; and the instruments or reagents without specified commercial sources are general products commercially available on the market.

### Example 1 Screening of the Components of Serum-Free Medium for Mesenchymal Stem Cells

### (1) Screening of the Content of Serum Substitute

Medium to be tested: 0.1 parts by volume of β-mercapto, 10 ng/ml recombinant human basic fibroblast growth factor (b-FGF, Peprotech), 1 part by volume of aqueous solution of non-essential amino acids (Catalog No. 11140, Gibco), 1, 2, 5, 8, 10, 12, 15, or 20 parts by volume of Knockout FBS serum substitute (Catalog No. 10828-028, Gibco), and 89 parts by volume of a-MEM.

In a biosafety cabinet, the third-passage hUC-MSCs separated from Wharton's jelly tissue of umbilical cord from a healthy neoborn by natural delivery were inoculated into a T75 cell culture flask at a density of 2×10⁴ cells/cm², and 12-15 ml of medium commercially available were added as well to culture the cells. The cells were cultured till cells were observed to have completely adhered to walls of the flask, and the medium was replaced with 15 ml of medium to be tested. The growth of the cells was observed.

Results:

The cells proliferated slowly in the medium comprising 1, 2 or 5 parts by volume of serum substitute respectively, and 24 hours after inoculation, it was observed that part of the hUC-MSCs gathered while the cells were flat with poor refractive index and about 20% confluence; and subsequent observation 48 hours after inoculation showed that the hUC-MSCs were bright but the proliferation was almost stopped when 60% confluence was reached (see panel 1A). However, the cells grew well in the medium comprising 8, 10 or 12 parts by volume of serum substitute respectively, and 24 hours after inoculation, it was observed that the hUC-MSCs appeared as spindle-shape and gathered in whorls spreading much more, and the cells were bright, and 40-60% confluence was reached; and subsequent observation 48 hours after inoculation showed that the hUC-MSCs were bright and more than 90% confluence was reached. Similar to those in the low concentrations of serum substitute, the cells proliferated slowly in the medium comprising 15 or 20 parts by volume of serum substitute respectively, and the cells were flat with a clear outline (see panel 1B).

(2) Screening the Content of Recombinant Human Basic Fibroblast Growth Factor

Medium to be tested: 0.1 parts by volume of β-mercapto ethanol, 1, 2, 5, 8, 10, 12, 15, 18, or 20 ng/ml recombinant human basic fibroblast growth factor (b-FGF, Peprotech), 1 part by volume of aqueous solution of non-essential amino acids (Catalog No. 11140, Gibco), 10 parts by volume of Knockout FBS serum substitute (Catalog No. 10828-028, Gibco), and 89 parts by volume of a-MEM.

With reference to the method described in part (1) above, the stem cells from the same source were inoculated at the same density, and cultured in 12-15 ml medium commercially available. The cells were cultured till cells were observed to have completely adhered to walls of the flask, the medium was replaced with 15 ml of medium to be tested. The growth of the cells was observed.

Results:

The cells proliferated slowly in the medium comprising 1 or 2 ng/ml bFGF respectively, were in poor and undernourished state (see panel 1C). The cells grew normally in the medium comprising 5, 8, 10, 12 or 15 ng/ml bFGF respectively, and the cells were bright and in a good state. The cells grew well and were bright in the medium comprising 18 or 20 ng/ml bFGF respectively; however after several passages, the cells were prone to differentiate, and formed into clusters, or had tentacles stretched out (see panel 1D).

### Example 2 Red Blood Cell Lysis Buffer Assisted Method of Extracting hUC-MSCs with Collagenase

Collecting and Transporting the Sample:

A umbilical cord sample from a newborn by natural delivery was collected aseptically, put into the preservation and transportion solution for umbilical cord comprising penicillin sodium, streptomycin sulfate, gentamicin and amphotericin B (the final concentration of each of penicillin sodium, streptomycin sulfate and gentamicin in D-Hank's was 150 U/ml respectively, while that of amphotericin B was 300 U/ml), then transported in ice to a clean GMP cell laboratory within 6 hours.

Washing and Sterilizing the Sample:

In a biosafety cabinet, the fresh umbilical cord sample was placed in a 50 ml aseptic centrifuge tube, washed 2 times with 75% aqueous ethanol, then washed 3 times with sterile saline.

Pre-Processing the Umbilical Cord Sample:

The umbilical cord was cut into segments each of about 2-3 cm in length with ophthalmic scissors, the intravascular blood was removed with tweezers. Then each segment was longitudinally cut, two umbilical arteries and one umbilical vein were eliminated with hemostatic forceps, and the Wharton's jelly tissue was bluntly dissected and put into a 50 ml centrifuge tube. PBS with a volume of 1.5-2 times the volume of the tissue was added, and the tissue was washed by shaking slightly.

Treating with the Red Blood Cell Lysis Buffer:

PBS washed Wharton's jelly tissue of umbilical cord was transferred to a 100 mm clean and sterile culture dish and cut into tissue blocks each in size of 1-3 mm³; the minced cord tissue was transferred into a centrifuge tube, and the red blood cell lysis buffer was added with a volume of 1.5-2 times the volume of the tissue blocks. The tissue blocks were treated with the buffer for 3 minutes at room temperature, the tissue blocks were collected by centrifugation at 1200 rpm, 4° C. for 6 minutes and the supernatant was discarded. The red blood cell lysis buffer comprised 5 g/L NH₄Cl and 0.1 mM Na₂-EDTA, pH7.2-7.4.

Digesting with Type IV Collagenase:

The digestion solution comprising type IV collagenase (D-Hank's comprising 1% type IV collagenase, 0.5% hyaluronidase, 300 U/ml DNase and 2% serum substitute) was added into the centrifuge tube with a volume of 2 times the volume of the tissue blocks, and the centrifuge tube was transferred into an incubator at 37° C., 5% CO₂. After 8-hour digestion, the centrifuge tube was removed out of the incubator and transferred to a clean bench. PBS was added to dilute, filtration was carried out through a 200 mesh sterile sieve, and the filtrate was collected to a 50 ml centrifuge tube, washed with PBS and centrifuged at 1200 rpm, 4° C. for 6 minutes to remove residual enzyme.

Primary Culturing hUC-MSCs:

The cell pellets on the bottom of the centrifuge tube were flicked, the serum-free medium for mesenchymal stem cells was added to give a cell suspension, and the cell suspension was incubated in 6 culture dishes of 100 mm at a density of 3×10⁴ cells/cm². The dishes were put into a constant temperature incubator for culture, during which the medium was replaced with fresh medium every 3 days. The serum-free medium for mesenchymal stem cells comprised 0.1 parts by volume of fβ-mercapto ethanol, 1 part by volume of aqueous solution of non-essential amino acids (Catalog No. 11140, Gibco), 10 parts by volume of Knockout™ serum substitute, 89 parts by volume of a-MEM/DMEM-F12 and b-FGF at a final concentration of 10 ng/ml.

On the 5^{th} day after culturing in the incubator, the supernatant of cell culture was slowly pipetted, and the followings were detected: hepatitis A, hepatitis B, hepatitis C, syphilis, human immunodeficiency virus, mycoplasma, chlamydia and endotoxin.

Cell Passage:

On the 6^{th} day of primary culture in the incubator while about 50% confluence was reached (see panel 2A), the cells were digested with 0.125% trypsin for 1-2 minutes (the side walls of culture dishes or flasks were patted during the digestion), then the cells were collected by centrifugation at 1200 rpm, 4° C. for 6 minutes and the supernatant was discarded. The cells were inoculated in a T75 cell culture flask in the ratio of 1:4 to continue passage till 90% confluence was reached, then the cells were collected for reserving or were further passage cultured.

The experimental results indicated that the purity of the hUC-MSCs reached above 99% after 2 passages. Panel 2B showed the fibrous morphology of cells cultured after passage.

### Example 3 Red Blood Cell Lysis Buffer Assisted Method of Extracting the hUC-MSC with Collagenase

The red blood cell lysis buffer used in this Example comprised 10 g/L NH₄Cl and 0.1 mM Na₂-EDTA, pH 7.2-7.4.

The process was carried out with reference to the method described in Example 2 above. The digestion was performed with the digestion solution comprising IV collagenase for 12 hours, and the primary cells were plated on 8 culture dishes. Mesenchymal stem cells began to adhere on the 2^{nd} day of the culture and 60% confluence was reached around the 7^{th} day, and the cells were passage cultured after trypsin digestion. The purity of the cells after three passages was greater than 99.2% while the viability was 99.7%.

### Example 4 Method of Extracting Mesenchymal Stem Cells without Using Red Blood Cell Lysis Buffer

Collecting, transporting, pre-processing and the like of the cord tissue sample were performed with reference to those described in Example 2 above. The Wharton's jelly tissue was cut into minced tissue blocks each in size of 1-3 mm³ and digested with the digestion solution comprising type IV collagenase for 8 hours without treatment with the red blood cell lysis buffer. The cells were evenly inoculated on 100 mm sterile culture dishes after the filtration performed through 200 mesh sieve, and 10 ml serum-free medium for mesenchymal stem cells was added to each dish. The dishes were transferred into an incubator at 37° C., 5% CO₂ for culture. On the 3^{rd} day of placement in the incubator, it was observed that some mesenchymal stem cells adhered; however, a large number of red blood cells adhered to the bottom of the dishes, occupying the area the stem cells should have adhered to, and the stem cells were in poor growth (see panel 2C).

### Example 5 Methods of Extracting Mesenchymal Stem Cells with Different Concentrations of Red Blood Cell Lysis Buffer

Collecting, transporting, and pre-processing and the like of the cord tissue sample were performed with reference to those described in Example 2 above. The Wharton's jelly tissue was cut into minced tissue blocks each in size of 1-3 mm³, and the red blood cell lysis buffer comprising 1, 2, 5, 7, 10, 15 or 20 g/L NH₄Cl and 0.1 mM Na₂-EDTA (pH 7.2-7.4) was added and treated the blocks for 2 minutes. Then the treated blocks were digested with the digestion solution comprising type IV collagenase for 8 hours, and the cells were evenly inoculated on 100 mm sterile culture dishes after the filtration performed through 200 mesh sieve, and 10 ml serum-free medium for mesenchymal stem cells was added to each dish. The dishes were transferred into an incubator at 37° C., 5% CO₂ for culture. The growth of the cells was observed.

Results:

Upon treatment with red blood cell lysis buffer comprising 1 or 2 g/L NH₄Cl, there were still evident red blood cells on the bottom of the culture dishes, preventing the mesenchymal stem cells from adherence. Upon treatment with red blood cell lysis buffer comprising 5, 7 or 10 g/L NH₄Cl, there were few red blood cells on the dish bottom, and the mesenchymal stem cells adhered quite quickly (2-3 days). Upon treatment with red blood cell lysis buffer comprising 15 or 20 g/L NH₄Cl, though no red blood cells were observed on the dish bottom, there were very few adherent mesenchymal stem cells but a large number of floating dead cells, and it took quite a long time for the stem cells to adhere (4-5 days).

### Example 6 Methods of Extracting Mesenchymal Stem Cells from Umbilical Cords with Different Duration of Treatment with Red Blood Cell Lysis Buffer

Collecting, transporting, pre-processing and the like of the cord tissue sample were performed with reference to those described in Example 2 above. The Wharton's jelly tissue was cut into minced tissue blocks each in size of 1-3 mm³, and red blood cell lysis buffer comprising 5 g/L NH₄Cl and 0.1 mM Na₂-EDTA (pH 7.2-7.4) was added and treated the cord tissue blocks for 1, 2, 5, 7 or 10 minutes respectively. Then the treated blocks were digested with the digestion solution comprising type IV collagenase for 8 hours, and the cells were evenly inoculated on 100 mm sterile culture dishes after the filtration performed through 200 mesh sieve, and 10 ml serum-free medium for mesenchymal stem cells was added to each dish. The dishes were transferred into an incubator at 37° C., 5% CO₂. The growth of the cells was observed.

Results:

Upon treatment for 1 minute, there were still evident red blood cells on the bottom of the culture dishes, preventing the mesenchymal stem cells from adherence. Upon treatment for 2 or 5 minutes, there were few red blood cells on the dish bottom, and a relatively larger number of mesenchymal stem cells adhered, and the cells were bright and adhered quite quickly (2-3 days). Upon treatment for 7 or 10 minutes, though no red blood cells were observed on the dish bottom, there were very few mesenchymal stem cells and the adherent cells grew very slowly.

### Example 7 Methods for Extracting Mesenchymal Stem Cells with Digestion Solutions Comprising Different Concentrations of Type IV Collagenase

Collecting, transporting, pre-processing and the like of the cord tissue sample were performed with reference to those described in Example 2 above. The Wharton's jelly tissue was cut into minced tissue blocks each in size of 1-3 mm³, and red blood cell lysis buffer comprising 5 g/L NH₄Cl and 0.1 mM Na₂-EDTA (pH 7.2-7.4) was added and treated the cord tissue blocks for 2 minutes. Then the treated tissue blocks were collected by centrifugation, and the digestion solution comprising type IV collagenase (D-Hank's comprising 0.1%, 0.5%, 1%, 2%, or 5% type IV collagenase, 0.5% hyaluronidase, 300 U/ml DNase and 2% serum substitute) was added into the centrifuge tube with a volume of 2 times the volume of the tissue blocks, and the centrifuge tube was transferred into an incubator at 37° C., 5% CO₂. After 8-hour digestion, the centrifuge tube was removed out of the incubator and transferred to a clean bench. Filtration was carried out through 200 mesh sterile sieve. Cell suspension was prepared by adding the serum-free medium for mesenchymal stem cells, and was incubated on 100 mm culture dishes at a density of 3×10 cells/cm². The dishes were put into an incubator at 37° C., 5% CO₂ for culture. The growth of the cells was observed.

Results:

After digestion for 8 hours with the digestion solution comprising 0.1% or 0.5% type IV collagenase, there were still a lot of undigested tissue blocks remained in the digestion solution, prone to block the 200 mesh sieve and made it difficult to perform the filtration. After digestion for 8 hours by the digestion solution comprising 1% type IV collagenase, the tissue blocks were digested completely with only a few undigested tissue blocks left, so the filtration was easy to perform with the sieve, and after plating most of the cells grew by adherence with only a few cells died. After digestion for 8 hours with the digestion solution comprising 2% or 5% type IV collagenase, the tissue blocks were digested completely; however when the cell were plated at the same density after filtration through 200 mesh sieve, massive cells died and were unable to grow by adherence.

### Example 8 Methods of Extracting Mesenchymal Stem Cells with Different Duration of Digestion with Type IV Collagenase

The experiment was carried out in the same conditions described in Example 5 above; however the digestion solution is the D-Hank's comprising 1% type IV collagenase, 0.5% hyaluronidase, 300 U/ml DNase and 2% serum substitute and the duration of digestion was 2 h, 4 h, 6 h, 8 h, 10 h, 12 h, 16 h, 20 h or 24 h respectively. The growth of the cells was observed.

Results:

After digestion for 2 h, 4 h or 6 h, the tissue blocks were digested incompletely with a large amount of undigested tissue blocks left in the digestion solution, prone to block the 200 mesh sieve and make it difficult to perform the filtration; and as a result, there were a low quantity of cells in the filtrate and the primary culture took quite a long time. After digestion for 8 h, 10 h or 12 h, the tissue blocks were digested completely with only a few undigested tissue blocks left, so the filtration was easy to perform with the sieve, and after plating most of the cells grew by adherence and with only a few cells died. After digestion for 16 h, 20 h or 24 h, the tissue blocks were digested completely; however, when the cell were plated at the same density after filtration through 200 mesh sieve, there were a relatively more dead cells unable to adhere, while the adherent cells grew slowly.

### Example 9 Morphological Identification of hUC-MSCs

By separation and culture as described in Example 3 above, bright and adherent cells in round shape were visible under microscope after 2 days of culture. After 3 days of culture, bright, adherent and round cells could be observed under microscope to have tentacles stretched out and adhered to the walls in spindle-shape. About 7 days, cell clusters growing in whorls appeared. During digestion and passage culture, the stem cells were in homogeneous morphology and proliferated rapidly, and state of the cells kept stable during passage.

### Example 10 Analysis of Surface Markers of hUC-MSCs by Flow Cytometry

The third-passage cells separated and cultured in Example 3 grew to 90% confluence, and digested with 2 ml 0.125% trypsin. The cells were collected by centrifugation at 1200 rpm, 4° C. for 6 minutes and the supernatant was discarded. After washed 2 times with PBS, the cells were transferred to flow cytometry tubes with 1×10⁵ cells in each tube. Then 5 μL of each of antibodies CD34-PE, CD45-FITC, CD29-FITC, CD44-PE, CD73-PE, CD105-PE, CD90-FITC, HLA-ABC-FITC, HLA-DR-PE, IgG1-PE (isotype control) and IgG-FITC (isotype control) was added into the tubes respectively, mixed and incubated in dark at 4° C. for 30 minutes. Afterwards, the cells were washed once with PBS, the supernatant was discarded by centrifugation, and the cells were re-suspended and mixed evenly in 500 μl of PBS, and then detected on a flow cytometer (Flow Cytometer XL, Beckman), with 1×10⁴ cells collected for each sample.

The immunophenotypes of the cells were as follows:

Positive expression: CD29>99.0%, CD44>99.0%, CD73>99.0%, CD105>99.0%, CD90>99.0%, and HLA-ABC 99.0%;

Negative expression: CD34<1.0%, CD45<1.0%, and HLA-DR<1.0%.

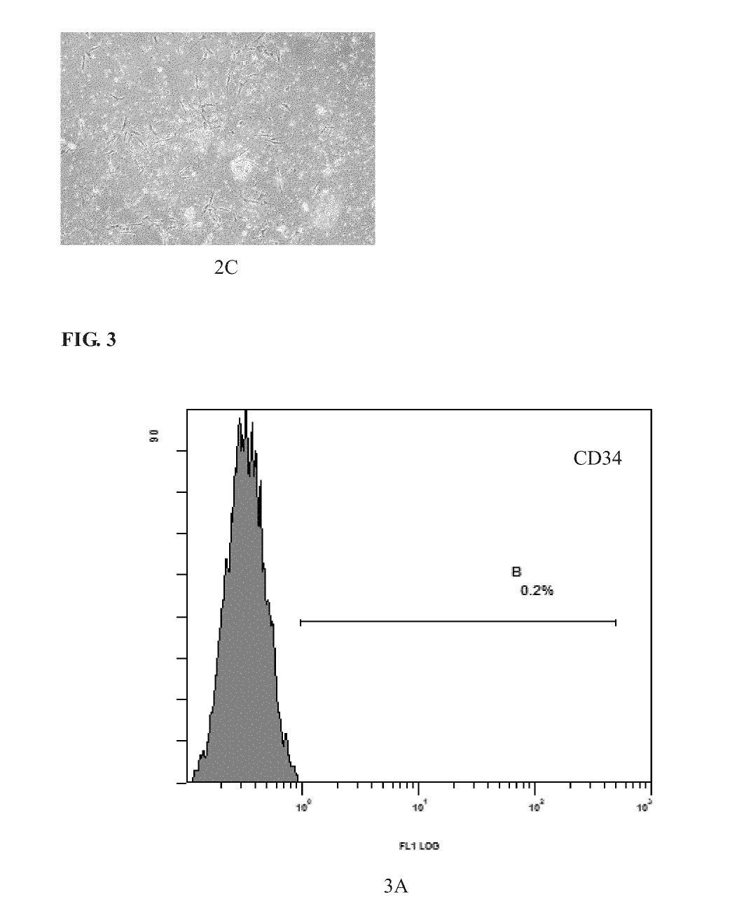

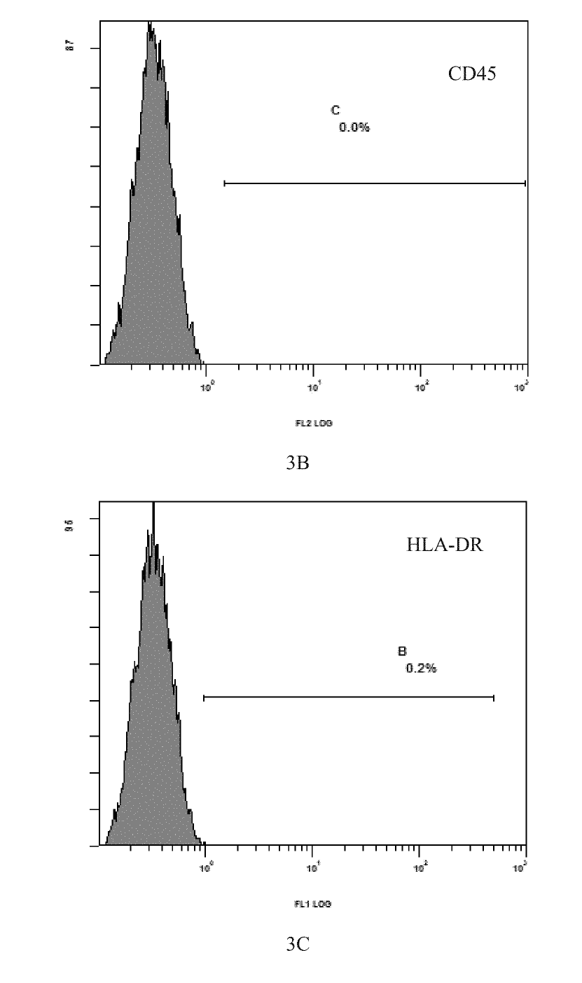

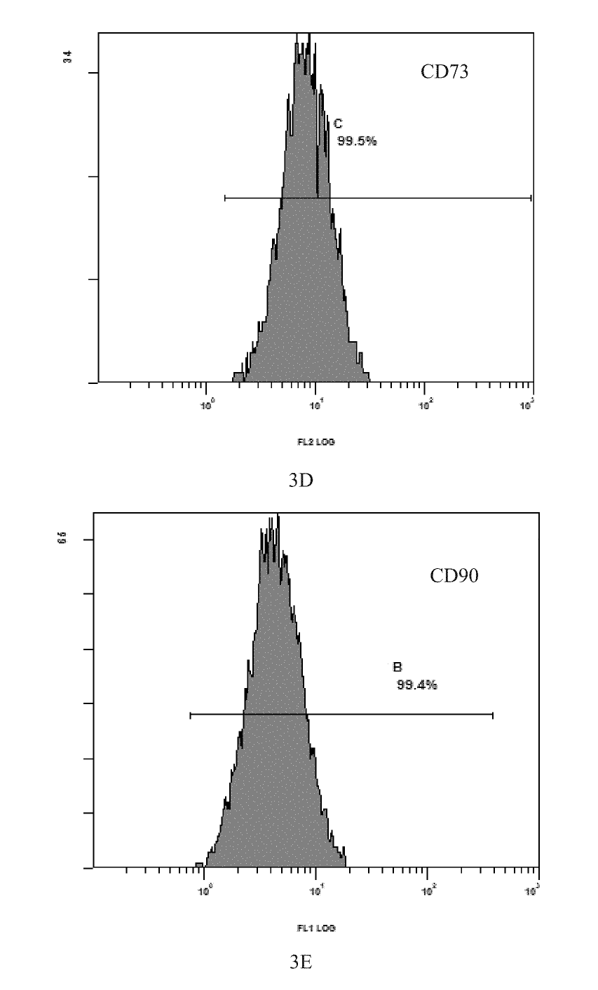

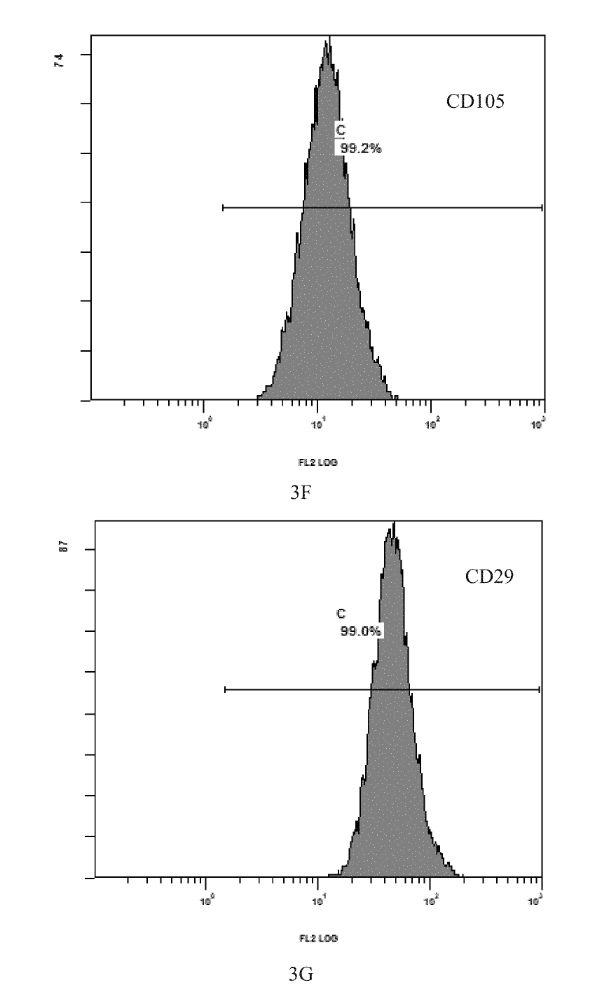

Results are provided in FIG. 3.

### Example 11 Analysis of Cell Viability and Growth Profile of hUC-MSCs by Cell Viability Analyzer

The second-passage cells separated and cultured in Example 3 were inoculated into a T25 culture flask. The cells grew to 90%-100% confluence, and were digested with 0.125% trypsin. Then the cells were collected and inoculated to two 6-well plates at a density of 1×10⁵ cells per well. After all the cells had adhered and partially grown for 10 hours, cells in two wells were collected and prepared into a cell suspension in 500 μl of PBS which was then analyzed on a cell viability analyzer (Cell Viability Analyzer Vi-Cell XR, Beckman). Then, sampling and analyzing were performed every 12 hours, and growth curve was drawn accordingly.

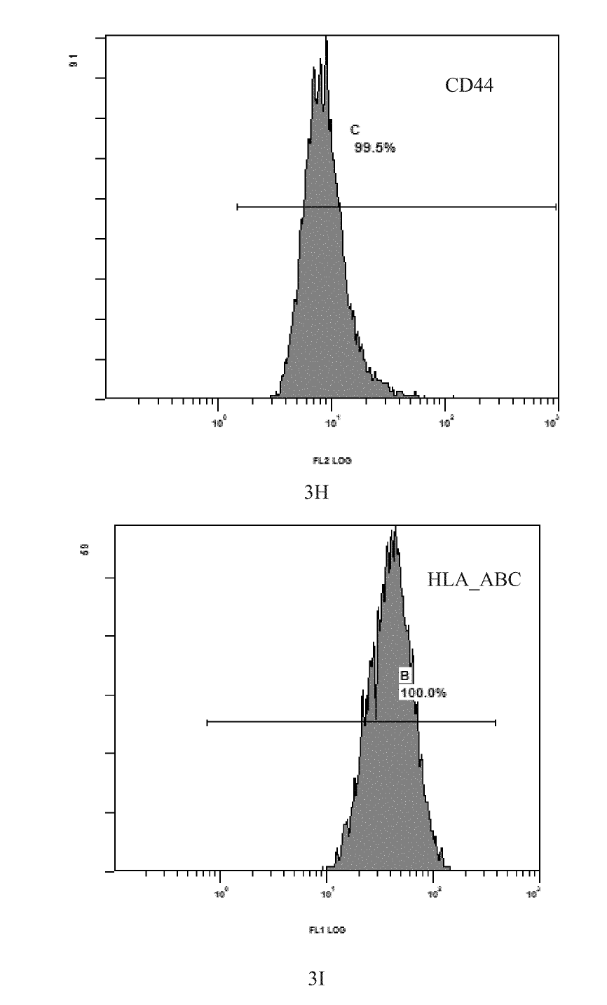

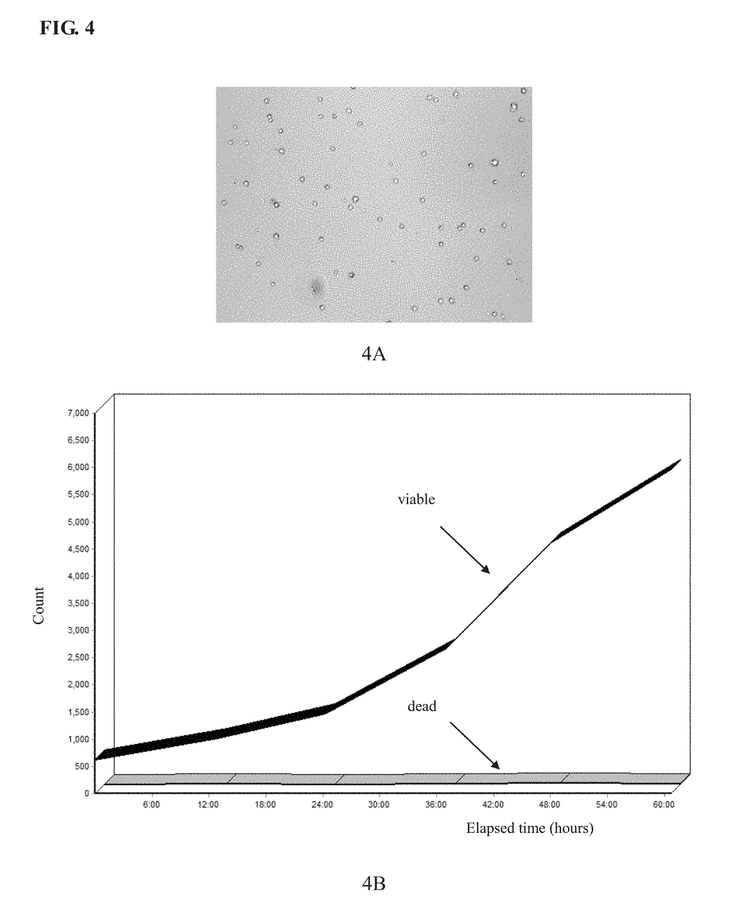

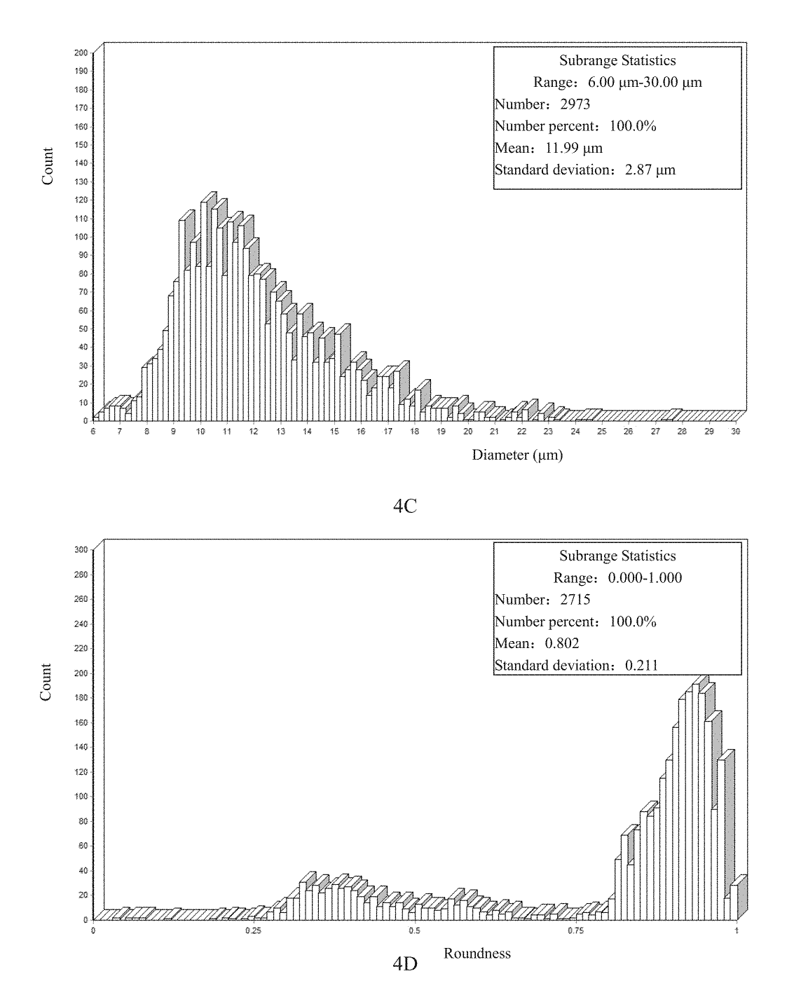

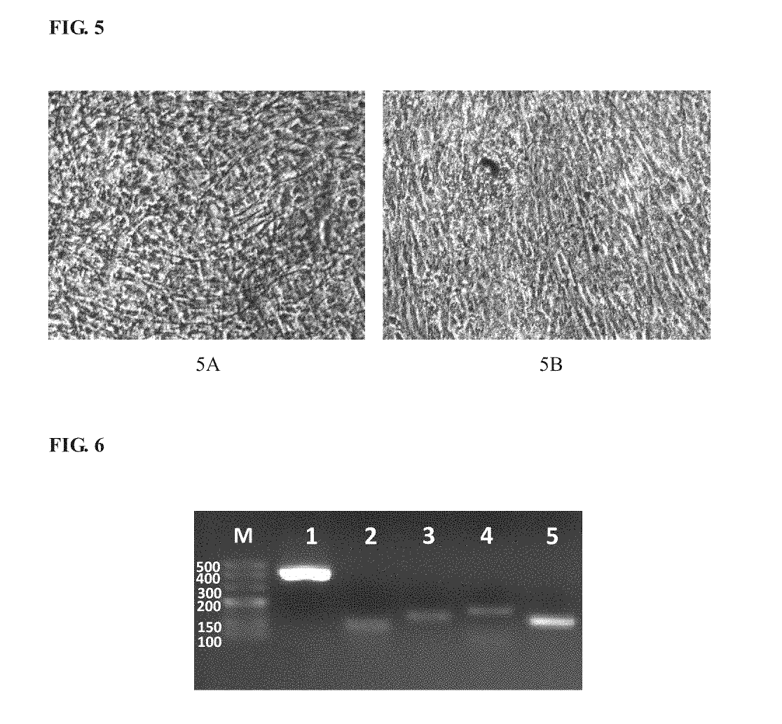

Results (see FIG. 4) showed that the viability of hUC-MSCs was above 99.7% with a diameter distribution of 9-12 μm. The hUC-MSCs which appeared as spindle-shape and grew in whorls had a perfect roundness after digestion, and a proliferation profile characterized by latent stage, logarithmic growth stage and platform stage.

### Example 12 Identification of Multi-Directional Differentiation Potential of hUC-MSCs

1) Osteogenic Differentiation

The fourth-passage hUC-MSCs separated and cultured in Example 2 were inoculated to a 6-well cell culture plate at a density of 3×10⁴ cells/cm². After 24 hours, 2 ml of freshly prepared human UC MSC osteogenic differentiation medium (HUXUC-90021, Cyagen) was added to each well, and the medium was replaced with fresh osteogenic differentiation medium every 3 days. After 2 weeks, the cells were fixed with paraformaldehyde, and stained with alizarin red for 3-5 minutes.

Results (see panel 5A) showed that alizarin red reacted with calcium nodules during osteogenesis to give a dark red color, after two weeks of osteogenic induction of the hUC-MSCs obtained by the method of present invention; furthermore, the osteogenic marker gene OPN also showed differential expression level before and after induction.

2) Adipogenic Differentiation

The fourth-passage hUC-MSCs separated and cultured in Example 2 were inoculated to a 6-well cell culture plate at a density of 2×10⁴ cells/cm². When 100% confluence had reached, fresh adipogenic differentiation medium A solution (HUXUC-90031, Cyagen) was added to each well to start induction; after 3 days, the medium was replaced with adipogenic differentiation medium B solution and the culture was maintained for 24 hours. The medium replacement happened in such a cycle. When more but fairly small fat droplets appeared, the culture system was maintained with adipogenic differentiation medium B solution for 7 days; and after induction, the cells were fixed with 4% paraformaldehyde and stained with oil red O.

Results (see panel 5B) showed that oil red O staining of adipogenic cells were obvious after two weeks of adipogenic induction of the hUC-MSCs prepared by the method of present invention.

### Example 13 RT-PCR Analysis of Pluripotency Genes of hUC-MSCs

The third-passage hUC-MSCs separated and cultured in Example 2 were inoculated to a T25 cell culture flask at a density of 5×10⁵ cells. 2-3 days later when 100% confluence had reached, the cells were collected, RNA extraction was performed by using total RNA extraction kit (R6834-01, OMRGA), and the extracted RNA was reverse transcribed by using reverse transcription kit (RR014A, TAKARA) to obtain cDNA sample which was then amplified by PCR. Then agarose gel electrophoresis was performed, and the results were observed by electrophoresis gel imager.

Results (see FIG. 6) showed that umbilical cord pluripotency marker genes NANOG, OCT4, SOX2 and SSEA4 had corresponding bands in different degrees of brightness.

### Example 14 Analysis of hUC-MSC Specific Proteins by Immunofluorescence Staining

The third-passage hUC-MSCs separated and cultured in Example 2 were inoculated to a 24-well cell culture plate at a density of 5×10³ cells per well. When 30%-50% confluence had reached, the cells were fixed with 4% paraformaldehyde for 15 minutes, and then were permeabilized with 0.25% Triton X-100 for 20 minutes. Subsequently, goat serum was used for blocking, diluted mouse anti-human primary antibody (anti-SOX2 antibody, anti-OCT4 antibody, anti-NANOG antibody or anti-NANOG antibody) was added and then the cells were incubated in dark at 4° C. over night. Then FITC labeled goat anti-mouse secondary antibody was added and the cells were incubated at room temperature for 2 hours. DAPI/PI was added to stain nuclei, and the cells were incubated in dark at room temperature for 20 minutes, and then were observed under a fluorescence microscope.

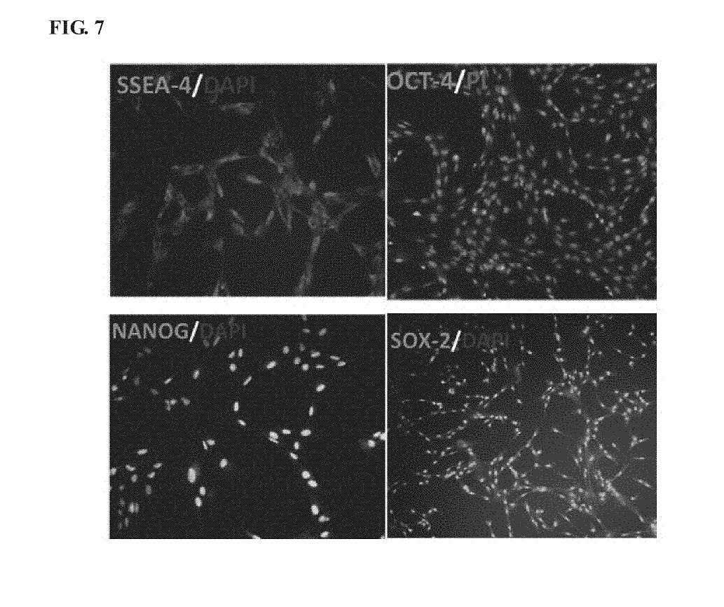

Results (see FIG. 7) indicated that hUC-MSCs separated and extracted by the method of the present invention expressed specific proteins SOX-2, OCT-4, NANOG and SSEA-4.

The above description of the specific implementations of the present invention is not to be construed as limiting the scope of the invention. The skilled person in this field can make various changes or deformations according to the present invention. As long as not departing from the spirit of the present invention, those changes or deformations are within the scope of the claims attached to the present invention.

1. A method for separating and extracting hUC-MSCs, the method comprising: using red blood cell lysis buffer and collagenase to treat Wharton's jelly tissue of umbilical cord.

2. The method according to claim 1, wherein the red blood cell lysis buffer is an aqueous solution comprising NH₄Cl and Na₂-EDTA, preferably an aqueous solution comprising 1-20 g/L NH₄Cl and 0.05-0.2 mM Na₂-EDTA, more preferably an aqueous solution comprising 5-10 g/L NH₄Cl and 0.1 mM Na₂-EDTA, pH 7.2-7.4;
preferably, the collagenase is type IV collagenase, preferably a digestion solution comprising type IV collagenase, more preferably D-Hank's comprising type IV collagenase, hyaluronidase, DNase and serum substitute, and further more preferably D-Hank's comprising 1% type IV collagenase, 0.5% hyaluronidase, 300 U/ml DNase and 2% serum substitute.

3. The method according to claim 1, wherein the method comprises: cutting the obtained Wharton's jelly tissue of umbilical cord into tissue blocks, adding the red blood cell lysis buffer with a volume of 1-3 times the volume of the tissue blocks, and treating the tissue blocks with the buffer at room temperature for 2-5 minutes; and, adding the digestion solution comprising type IV collagenase into the treated tissue blocks for further treatment.

4. The method according to claim 1, wherein the method further comprises: after digestion by collagenase, culturing with serum-free medium for mesenchymal stem cells to obtain primary mesenchymal stem cells; wherein the serum-free medium for mesenchymal stem cells comprises a-MEM/DMEM-F12, β-mercapto ethanol, non-essential amino acids, recombinant human basic fibroblast growth factor (b-FGF) and serum substitute;
preferably, the serum-free medium for mesenchymal stem cells comprises 0.05-0.2 parts by volume of β-mercapto ethanol, 0.5-2 parts by volume of an aqueous solution of non-essential amino acids, 8-12 parts by volume of serum substitute, 85-95 parts by volume of a-MEM/DMEM-F12 and recombinant human basic fibroblast growth factor at a final concentration of 5-15 ng/ml, wherein the aqueous solution of non-essential amino acids comprises glycine, alanine, L-asparagine, L-aspartic acid, glutamic acid, proline and serine each at a concentration of 8-12 mM;
more preferably, the serum-free medium for mesenchymal stem cells comprises 0.1 parts by volume of β-mercapto ethanol, 1 part by volume of the aqueous solution of non-essential amino acids, 10 parts by volume of the serum substitute, 89 parts by volume of a-MEM/DMEM-F12 and the recombinant human basic fibroblast growth factor at a final concentration of 10 ng/ml;
most preferably, the serum-free medium for mesenchymal stem cells consists of a-MEM/DMEM-F12, β-mercapto ethanol, the aqueous solution of non-essential amino acids, the recombinant human basic fibroblast growth factor and the serum substitute.

5. The method according to claim 1, wherein the method comprises: cutting the obtained Wharton's jelly tissue of umbilical cord into tissue blocks each in size of 1-3 mm³, adding the red blood cell lysis buffer with a volume of 1.5-2 times the volume of the tissue blocks, and treating the tissue blocks with the buffer at room temperature for 2-5 minutes; and into the treated tissue blocks, adding the digestion solution comprising type IV collagenase with a volume of 2-3 times the volume of the treated tissue blocks, and performing digestion at 37° C., 5% CO₂ for 8-12 hours;
preferably, the method of the present invention further comprises: inoculating obtained cells in the serum-free medium for mesenchymal stem cells at a density of 1-5×10⁴ cells/cm² culture dish area and culturing at 37° C., 5% CO₂, during which the medium is replaced with fresh medium every 3-4 days.

6. The method according to claim 1, wherein the method comprises the following steps:
(1) Pretreatment of umbilical cord tissue:
cutting fresh umbilical cord into segments, removing intravascular blood, longitudinally cutting each segment, eliminating umbilical artery and umbilical vein, bluntly dissecting the Wharton's jelly tissue and washing the same with PBS;
(2) Treatment with the red blood cell lysis buffer:
cutting the Wharton's jelly tissue obtained by step (1) into tissue blocks, adding the red blood cell lysis buffer to treat the tissue blocks, collecting the treated tissue blocks by centrifugation and washing them with PBS;
(3) Digestion with collagenase:
adding the digestion solution comprising type IV collagenase into the treated tissue blocks obtained by step (2) for further treatment, diluting by adding PBS, and collecting cells by filtration through sterile sieve;
(4) Primary culture:
culturing the cells obtained by step (3) in the serum-free medium for mesenchymal stem cells to obtain primary mesenchymal stem cells;
preferably, the method further comprises the following steps:
(5) Supernatant detection:
taking the supernatant of cell culture obtained by step (4), and detecting one or more selected from the group consisting of hepatitis A, hepatitis B, hepatitis C, syphilis, human immunodeficiency virus, mycoplasma, chlamydia and endotoxin;
(6) Passage culture:
selecting the cultured cells with negative results in step (5), collecting cells by centrifugation after trypsin digestion, passage culturing, and collecting the cells for reserving or freezing conservation, or continuing passage;
(7) Cell detection:
taking the cells cultured in step (6), and detecting one or more selected from the group consisting of differentiation, cell activity, cell purity, cell contamination and proliferation profile.

7. The method according to claim 1, wherein the step (1) includes: cutting the fresh umbilical cord into segments each of 2-3 cm in length, removing the intravascular blood, longitudinally cutting each segment, eliminating the umbilical artery and the umbilical vein, bluntly dissecting the Wharton's jelly tissue, adding PBS with a volume of 1.5-2 times the volume of the tissue, and washing the tissue by shaking slightly;
preferably, the step (2) includes:
cutting the washed Wharton's jelly tissue of umbilical cord into tissue blocks each in size of 1-3 mm³, adding the red blood cell lysis buffer with a volume of 1.5-2 times the volume of the tissue blocks, treating the tissue blocks with the buffer at room temperature for 2-5 minutes, collecting the treated tissue blocks by centrifugation, and washing 2-3 times with PBS; wherein the centrifugation preferably is performed at 1000-1200 rpm, 4° C. for 6 minutes;
preferably, the step (3) includes:
adding the digestion solution comprising type IV collagenase with the volume of 2-3 times the volume of the treated tissue blocks, performing digestion at 37° C., 5% CO₂ for 8-12 hours, and diluting by adding PBS; and, filtering through a 100 mesh or 200 mesh sterile sieve, collecting filtrate, washing with PBS, and performing centrifugation to obtain cells; wherein preferably, the centrifugation is performed at 1000-1200 rpm, 4° C. for 6 minutes;
preferably, the step (4) includes:
inoculating the cells obtained by step (3) in the serum-free medium for mesenchymal stem cells at a density of 1-5×10⁴ cells/cm² culture dish area and culturing at 37° C., 5% CO₂, during which the medium is replaced with fresh medium every 3-4 days;
preferably, the step (5) includes:
taking the supernatant of cell culture obtained by step (4) and detecting all of the followings: hepatitis A, hepatitis B, hepatitis C, syphilis, human immunodeficiency virus, mycoplasma, chlamydia and endotoxin;
preferably, the step (6) includes:
Selecting the cultured cells with all negative results in step (5), performing trypsin digestion at 30-60% confluence, collecting cells by centrifugation, passage culturing to reach above 90% confluence, and collecting the cells for freezing conservation or continuing passage culture; wherein preferably, the trypsin is used at a mass percent concentration of 0.125%, and the digestion is performed for 1-2 minutes while patting the side walls of culture dish or culture flask used; and wherein preferably, the centrifugation is performed at 1000-1200 rpm, 4° C. for 6 minutes;
preferably, the collected cells are freezing conserved in liquid nitrogen at −196° C. at a density of 2-3×10⁶ cells/ml; or preferably, the collected cells are passage cultured in a rate of 1:3-1:4;
preferably, the step (7) includes:
taking the cells cultured in step (6), and detecting all of the followings: differentiation, cell activity, cell purity, cell contamination and proliferation profile.

8. The method according to claim 1, wherein, prior to the step (1), the method further comprises washing, preserving and pre-processing the umbilical cord;
preferably comprising:
collecting aseptically umbilical cord tissue from a healthy newborn by natural or cesarean section delivery, putting the umbilical cord tissue into preservation and transportion solution for umbilical cord after surface washing with sterile saline, preferably transporting the umbilical cord tissue in ice to a clean cell laboratory within 6 hours; and before use, washing the fresh umbilical cord 2-3 times with 75% aqueous ethanol, then 3-5 times with sterile saline;
preferably, the preservation and transportion solution for umbilical cord is magnesium and calcium free D-Hank's comprising penicillin sodium, streptomycin sulfate, gentamicin and amphotericin B for injection; more preferably, the concentration of each of penicillin sodium, streptomycin sulfate and gentamicin is 100-200 U/ml, preferably 150 U/ml; and the concentration of amphotericin B is 200-400 U/ml, preferably 300 U/ml.

9. hUC-MSCs prepared by the method according to claim 1.

10. The hUC-MSCs according to claim 9, wherein the mesenchymal stem cells have characteristics as follows:
(1) adhering to plastic container(s), appearing as spindle-shape and growing in whorls;
(2) ratio of CD29, CD44, CD73, CD90, CD105 or HLA-ABC positive cells greater than 99.0%; and ratio of CD45, CD34 or HLA-DR positive cells less than 1.0%;
(3) capable of being induced to differentiate into osteogenic cells and adipogenic cells in vitro;
(4) ratio of viable cells detected above 99%;
(5) having a typical “S type” growth curve; and
(6) expressing pluripotency genes which are one or more selected from the group consisting of SSEA-4, OCT-4, NANOG and SOX-2.

11. Use of the red blood cell lysis buffer and/or the digestion solution comprising type IV collagenase as defined in claim 2 in the preparation of an agent for separating and culturing mesenchymal stem cells.

12. A kit for separating and culturing mesenchymal stem cells, wherein the kit comprises the red blood cell lysis buffer and/or the digestion solution comprising type IV collagenase as defined in claim 2.

13. Use of the serum-free medium for mesenchymal stem cells as defined claim 4 in the preparation of an agent for separating and culturing mesenchymal stem cells.

14. A kit for separating and culturing mesenchymal stem cells, wherein the kit comprises the serum-free medium for the mesenchymal stem cells as defined in claim 4.

15. Use of the red blood cell lysis buffer and/or the digestion solution comprising type IV collagenase and the serum-free medium for mesenchymal stem cells as defined claim 4 in the preparation of an agent for separating and culturing mesenchymal stem cells.

16. A kit for separating and culturing mesenchymal stem cells, wherein the kit comprises the red blood cell lysis buffer and/or the digestion solution comprising type IV collagenase and the serum-free medium for the mesenchymal stem cells as defined in claim 4.
